(19) United States
(12) Patent Application Publication
(10) Pub. No.: US 2022/0315502 A1
(43) Pub. Date: Oct. 6, 2022

(54) LIQUID FERTILIZER PRODUCTION METHOD AND HIGHQUALITY LIQUID FERTILIZER BASED ON L F Q C AND CHLORELLA MICROBIOLOGICAL FERTILIZER MANUFACTURE METHOD

(71) Applicant: Myung Gyu LEE, Wonju-si, Gangwon-do (KR)

(72) Inventors: Myung Gyu LEE, Wonju-si, Gangwon-do (KR); Sung Ha HONG, Wonju-si, Gangwon-do (KR); Soo Ryang KIM, Icheon-si, Gyeonggi-do (KR); Tak Won KANG, Wonju-si, Gangwon-do (KR)

(73) Assignee: Myung Gyu LEE, Wonju-si, Gangwon-do (KR)

(21) Appl. No.: 17/431,346

(22) PCT Filed: Jan. 10, 2020
(86) PCT No.: PCT/KR2020/000461

§ 371 Date: Apr. 20, 2022

(30) Foreign Application Priority Data
Feb. 15, 2019 (KR) 10-2019-0017589

(51) Int. Cl. C05F 17/40; C12N 1/12

(52) U.S. Cl. CPC C05F 17/40; C12N 1/12

(57) ABSTRACT

A method for producing a liquid fertilizer based on liquid fertilizer quality certification (LFQC) of livestock manure, includes: subjecting a liquid released from livestock manure to high-temperature aerobic liquid-phase fermentation; subjecting the liquid resulting from the high-temperature aerobic liquid-phase fermentation to post-curing fermentation to produce a fermentation liquid fertilizer that complies with the standards of liquid fertilizer quality certification (LFQC) of livestock manure; and subjecting the produced fermentation liquid fertilizer to a separation membrane to produce a high-quality liquid fertilizer with reduced solid particles.

### CROSS-REFERNECE TO PRIOR APPLICATIONS

This application is a National Stage Patent Application of PCT International Patent Application No. PCT/KR2020/000461 (filed on Jan. 10, 2020) under 35 U.S.C. § 371, which claims priority to Korean Patent Application No. 10-2019-0017589 (filed on Feb. 15, 2019), which are all hereby incorporated by reference in their entirety.

### BACKGROUND

The present disclosure relates to a technology for producing a high-quality liquid fertilizer based on the liquid fertilizer quality certification (LFQC) of livestock manure, and more specifically, to a method for producing a liquid fertilizer based on the liquid fertilizer quality certification (LFQC) of livestock manure, in which a high-quality liquid fertilizer that complies with the standards of liquid fertilizer quality certification (LFQC) of livestock manure is produced, and a microbial fertilizer containing Chlorella cultured using the produced high-quality liquid fertilizer as a medium; a high-quality liquid fertilizer produced by the method, and a method for preparing a Chlorella microbial fertilizer.

The use of fertilizers through the conversion of livestock manure into resources has a potential for livestock disease caused by pathogenic microorganisms in the manure, secondary contamination of agricultural products, and consumer safety issues.

Therefore, the management of livestock manure should thoroughly consider not only environmental and resource factors, but also hygiene and quarantine aspects throughout the entire process ranging from manure management in the livestock house to a treatment process, transportation, and land application.

In Korea, since the late 1980s, a system that can prepare a high-quality liquid fertilizer through fermentation of livestock manure at high temperature by utilizing the characteristics of a high-temperature aerobic liquid-phase fermentation technology for livestock manure has been continuously developed.

The high-temperature aerobic liquid-phase fermentation technology refers to a technology in which a high-temperature fermentation process is maintained by preservation and utilization of the heat generated in the aerobic metabolism process generated when thermophilic aerobic microorganisms digest organic matter, the aeration, and stirring heat of a fermentation device within the fermentation device.

The key to this technology is to maintain the growth of microorganisms to a maximum level by appropriately adjusting the oxygen supply method. The high-temperature aerobic digestion method mainly utilizes thermophilic microorganisms, and it is very important to maintain the activity at 55° C. to 65° C. where the activity is strong.

The greatest advantage of such a high temperature condition is that harmful factors can be controlled in the treatment process by inactivating pathogenic microorganisms and viruses present in the manure. In addition, by promoting the growth of thermophilic aerobic microorganisms, volatile fatty acids (VFA) and hydrogen sulfide (H₂S), which are odor-causing materials, are efficiently decomposed, thus making it possible to produce a clean fermentation liquid (a liquid fertilizer) with secured availability and safety.

As an example, the “method for treatment of livestock excrements using thermophilic aerobic fermentation, lime solidification, and separation by reverse osmosis membrane” of Korean Patent Publication No. 10-0747682 (Aug. 2, 2007) relates to a method for treating livestock excrement comprising a high-temperature aerobic fermentation step, a chemical coagulation process step using lime, and a physical purification process step using by reverse osmosis membrane separation, in which in the high-temperature aerobic fermentation step, livestock excrement passes through the excrement storage tank and high-temperature aerobic fermentation device and then discharged to a fermentation liquid storage tank, and the chemical coagulation process step using lime consists of steps in which the materials stored in the fermentation liquid storage tank through the high-temperature aerobic fermentation step are passed through a mixing tank, a primary flocculation tank, a dehydration device, a secondary flocculation tank, and a dehydration device, and are then discharged to a filtrate storage tank, and the physical purification process step using the reverse osmosis membrane separation consists of passing through a reverse osmosis membrane separation device and a concentrate storage tank, thereby promoting the removal of pathogenic microorganisms and decomposition of organic matter, and producing lime treatment fertilizer using a high-temperature aerobic fermentation device operated at a high temperature between 50° C. and 60° C., improving the efficiency of the physical purification process by removing nitrogen, phosphorus, SS, color and organic matter, and concentrating the residual organic matter and nitrogen and producing a liquid fertilizer thereby converting livestock excrement into resources.

In another example, the “apparatus for offensive odor treatment of high-temperature aerobic fermentation system” of Korean Patent Publication No. 10-0877588 (Dec. 30, 2008) is configured such that, when treating animal/plant organic waste (e.g., food waste, municipal sewage sludge, manure, etc.) via high-temperature liquid fermentation, a pipe is connected between an outlet and air inlets of the high-temperature liquid fermentation device to allow odor-generating gases (e.g., ammonia, etc.) to be circulated internally rather than being discharged to the outside by connecting a bio-filter at one side or both sides for filtration of a small amount of gas components discharged to the outside through the circulation pipe, thus capable of simply removing odors even without installing complex and cumbersome odor removing facilities and significantly reducing the amount of gas to be treated by the biofilter to thereby increase the adsorption filtration efficiency.

Meanwhile, the current legal standard for liquid fertilizer components refers to “fermentation liquid of livestock manure” among the “By-Product Fertilizers” of “Fertilizer Management Act”, and “Setting Up and Designation of Fertilizer Processing Standards”.

In this standard, the content of the standards (a total amount of nitrogen (N)-phosphate (P)-potassium (K) components), the maximum amount of harmful components to be contained (eight kinds of heavy metals and two pathogenic microorganisms), and other specifications (salts and moisture contents), etc. are presented.

In general, when a liquid fertilizer is used for livestock, a precautionary measure should be taken with respect to public complaints about odors, damage to crops due to overspray, the spread of harmfulness due to residual pathogens, etc., and as an alternative, maturing conditions (quality standards) of a liquid fertilizer are being demanded.

However, no specific quality standards are available for physicochemical properties other than fertilizer components (nitrogen-phosphate-potassium), the degree of maturation of liquid fertilizer infertility, etc. in the specifications of “fermentation liquid of livestock manure” in accordance with the “Setting Up and Designation of Fertilizer Processing Standards”, self-farming farmhouses are avoiding the use of the same due to production of a liquid fertilizer with low quality.

Therefore, for the wide-area use of a liquid fertilizer, according to the current “Fertilizer Management Act”, it is necessary to prepare a system that can be distributed and commercialized for various purposes by inducing realization of a high-quality liquid fertilizer other than “fermentation liquid of livestock manure”, and various system improvement R&D, such as “liquid fertilizer quality certification (LFQC) of livestock manure”, is currently in progress.

In an example, Korean Patent Publication No. 10-1859167 (May 11, 2018) relates to “a method for measuring maturity degree of liquid fertilizer of livestock manure”, in which a sample for maturity degree of a liquid fertilizer is collected, and while the maturity degree of the collected liquid fertilizer is measured by the seed germination rate of the pretreatment solution to dilute the collected liquid fertilizer sample in connection with the measurement of gas concentration and chromaticity of the collected liquid fertilizer sample, the liquid fertilizer sample is diluted by calculating the moisture content for the amount of manure and the dilution ratio of the raw materials according to the target moisture as the average value, and calculating the dilution ratio of the sample according to the solid content, thereby making it possible to accurately measure whether the liquid fertilizer of livestock manure has been matured.

### SUMMARY

In an embodiment, the present disclosure provides a method for producing a liquid fertilizer based on the liquid fertilizer quality certification (LFQC) of livestock manure that complies with the proposed standards of liquid fertilizer quality certification (LFQC) of livestock manure, a high-quality liquid fertilizer produced through the same, and a method for preparing a Chlorella microbial fertilizer.

In an embodiment, the present disclosure provides a method for producing a liquid fertilizer based on the liquid fertilizer quality certification (LFQC) of livestock manure, which can produce a liquid fertilizer in which high-concentration nitrogen is enriched; a high-quality liquid fertilizer produced through the same; and a method for preparing a Chlorella microbial fertilizer.

In an embodiment, the present disclosure provides a method for producing a liquid fertilizer based on the liquid fertilizer quality certification (LFQC) of livestock manure, which can prepare a microbial fertilizer containing Chlorella cultured using a high-quality liquid fertilizer as a medium that complies with the proposed standards of liquid fertilizer quality certification (LFQC) of livestock manure; a high-quality liquid fertilizer produced through the method; and a method for preparing a Chlorella microbial fertilizer.

Among the embodiments, the method for producing a liquid fertilizer based on the liquid fertilizer quality certification (LFQC) of livestock manure includes subjecting a liquid released from livestock manure to high-temperature aerobic liquid-phase fermentation; subjecting the liquid resulting from the high-temperature aerobic liquid-phase fermentation to post-curing fermentation to produce a fermentation liquid fertilizer that complies with the standards of liquid fertilizer quality certification (LFQC) of livestock manure; and subjecting the produced fermentation liquid fertilizer to a separation membrane to produce a high-quality liquid fertilizer with reduced solid particles.

The method for producing a liquid fertilizer may further include a step of bringing the ammonia gas, which is produced in the step of the high-temperature aerobic liquid-phase fermentation, into contact with water in an ammonia capturing tank to thereby capture ammonia.

In the step of capturing ammonia gas, phosphoric acid with a purity of 85% may be injected into the ammonia capturing tank at 1% of a treatment capacity, and thereby the ammonia gas may be recovered by capturing and concentrating with a phosphoric acid solution.

The method for producing a liquid fertilizer may further include a step of mixing the fermentation liquid fertilizer and the ammonia captured solution at a 1:1 ratio to thereby produce a liquid fertilizer in which high-concentration nitrogen is enriched.

Among the embodiments, the high-quality liquid fertilizer produced through the method for producing a liquid fertilizer based on the liquid fertilizer quality certification (LFQC) of livestock manure may be produced through the method for producing a liquid fertilizer.

Among the embodiments, the method for preparing a Chlorella microbial fertilizer includes subjecting a liquid released from livestock manure to high-temperature aerobic liquid-phase fermentation, and capturing the ammonia gas produced in the step of the high-temperature aerobic liquid-phase fermentation; subjecting the liquid resulting from the high-temperature aerobic liquid-phase fermentation to post-curing fermentation, subjecting the produced fermentation liquid fertilizer to a separation membrane to produce a high-quality liquid fertilizer that complies with the standards of liquid fertilizer quality certification (LFQC) of livestock manure; mixing the high-quality liquid fertilizer produced and the ammonia captured solution to prepare a mixed fermentation medium; and culturing Chlorella in the mixed fermentation medium to prepare a Chlorella microbial fertilizer.

In the step of preparing the mixed fermentation medium, the prepared mixed fermentation medium may be diluted and a medium for culturing Chlorella to a total-nitrogen (T-N) concentration corresponding to BG11 chemical medium may be prepared.

The disclosed technology may have the following effects. However, it does not mean that a specific embodiment should include all of the following effects or only the following effect, therefore, it should not be understood that the scope of the disclosed technology is limited by these embodiments.

The method for producing a high-quality liquid fertilizer based on the liquid fertilizer quality certification (LFQC) of livestock manure according to an embodiment of the present disclosure, the high-quality liquid fertilizer produced through the same, and the method for preparing a Chlorella microbial fertilizer can produce a liquid fertilizer that complies with the proposed standards of liquid fertilizer quality certification (LFQC) of livestock manure by subjecting a liquid released from livestock manure to high-temperature aerobic liquid-phase fermentation; and subjecting the liquid resulting from the high-temperature aerobic liquid-phase fermentation to post-curing fermentation.

The method for producing a high-quality liquid fertilizer based on the liquid fertilizer quality certification (LFQC) of livestock manure according to an embodiment of the present disclosure, the high-quality liquid fertilizer produced through the same, and the method for preparing a Chlorella microbial fertilizer can produce a liquid fertilizer with an improved quality to the next level, by producing a liquid fertilizer that complies with the proposed standards of liquid fertilizer quality certification (LFQC) of livestock manure, and reducing solid particles by subjecting the produced liquid fertilizer to a process of separation membrane treatment.

The method for producing a high-quality liquid fertilizer based on the liquid fertilizer quality certification (LFQC) of livestock manure according to an embodiment of the present disclosure, the high-quality liquid fertilizer produced through the same, and the method for preparing a Chlorella microbial fertilizer can produce a high-concentration-nitrogen-enriched liquid fertilizer in which the nitrogen concentration is increased one level higher, by mixing the ammonia-captured solution captured in the high-temperature aerobic liquid-phase fermentation process with the liquid fertilizer produced to comply with the proposed standards of liquid fertilizer quality certification (LFQC) of livestock manure.

The method for producing a high-quality liquid fertilizer based on the liquid fertilizer quality certification (LFQC) of livestock manure according to an embodiment of the present disclosure, the high-quality liquid fertilizer produced through the same, and the method for preparing a Chlorella microbial fertilizer can produce a Chlorella microbial fertilizer, by preparing a mixed fermentation medium that can replace the high-cost chemical medium for producing chlorella by mixing the ammonia-captured solution captured in the high-temperature aerobic liquid-phase fermentation process with the high-quality liquid fertilizer produced to comply with the proposed standards of liquid fertilizer quality certification (LFQC) of livestock manure, followed by culturing Chlorella in the prepared mixed fermentation medium. Accordingly, it is possible to produce a Chlorella microbial fertilizer without using an expensive chemical medium, thereby reducing crop production costs.

### BRIEF DESCRIPTION OF THE DRAWINGS

FIGS. 1A and 1B show drawings illustrating a liquid fertilizer production system based on the liquid fertilizer quality certification (LFQC) of livestock manure according to an embodiment of the present disclosure.

FIG. 2 shows a chart illustrating a process for producing a liquid fertilizer based on the liquid fertilizer quality certification (LFQC) of livestock manure according to an embodiment of the present disclosure.

FIG. 3 shows a chart illustrating a process for preparing a Chlorella microbial fertilizer according to an embodiment of the present disclosure.

FIG. 4 shows a graph illustrating the change in density of Chlorella cultured cells in a mixed fermentation medium and a chemical medium.

FIG. 5 shows a graph illustrating the results of the germination experiment of pepper seeds treated with a Chlorella microbial fertilizer.

### DETAILED DESCRIPTION

In a most preferred embodiment to implement the invention, the present disclosure provides a method for producing a liquid fertilizer based on liquid fertilizer quality certification (LFQC) of livestock manure, which includes: subjecting a liquid released from livestock manure to high-temperature aerobic liquid-phase fermentation; subjecting the liquid resulting from the high-temperature aerobic liquid-phase fermentation to post-curing fermentation to produce a fermentation liquid fertilizer that complies with the standards of liquid fertilizer quality certification (LFQC) of livestock manure; and subjecting the produced fermentation liquid fertilizer to a separation membrane to produce a high-quality liquid fertilizer with reduced solid particles; and a high-quality liquid fertilizer produced through the method.

In a most preferred embodiment to implement the invention, the present disclosure provides a method for preparing a Chlorella microbial fertilizer, which includes: subjecting a liquid released from livestock manure to high-temperature aerobic liquid-phase fermentation, and capturing the ammonia gas produced in the step of the high-temperature aerobic liquid-phase fermentation; subjecting the liquid resulting from the high-temperature aerobic liquid-phase fermentation to post-curing fermentation, and subjecting the produced fermentation liquid fertilizer to a separation membrane to produce a high-quality liquid fertilizer that complies with the standards of liquid fertilizer quality certification (LFQC) of livestock manure; mixing the high-quality liquid fertilizer produced and the ammonia captured solution to prepare a mixed fermentation medium; and culturing Chlorella in the mixed fermentation medium to prepare a Chlorella microbial fertilizer.

Since the description of the present disclosure merely provides embodiments for structural or functional explanations, the scope of the present disclosure should not be construed as being limited by these embodiments described in the specification. That is, since these embodiments can have various changes and various forms, it should be understood that the scope of the present disclosure includes equivalents thereof that can realize the technical idea. In addition, since the purposes or effects presented in the present disclosure do not mean that a specific embodiment should include all of them or only such an effect, the scope of the present disclosure should not be construed as being limited thereby.

Meanwhile, the meaning of the terms described in the present disclosure should be understood as follows.

Terms such as “first” and “second” are for distinguishing one component from another, and the scope of rights should not be limited by these terms. For example, a first component may be termed a second component, and similarly, a second component may also be termed a first component.

When a component is referred to as “being connected” to another component, it may be directly connected to the other component, but it should also be understood that another component may be interposed therebetween. In contrast, when it is described that a certain component is “being directly connected” to another element, it should be understood that the other component does not exist therebetween. Meanwhile, other expressions describing the relationship between components, that is, “between” and “immediately between” or “adjacent to” and “directly adjacent to”, etc., should be likewise interpreted in the same way.

A singular expression is to be understood to include a plural expression unless the context clearly indicates otherwise; terms such as “comprise (include)” or “have” are intended to designate the presence of an implemented feature, a number, a step, an action, a component, a constituent, or a combination thereof; and it should be understood that these do not preclude the possibility of existence or addition of one or more other features or numbers, steps, actions, components, constituents, or combinations thereof.

In each step, the identification number (e.g., a, b, c, etc.) is used for convenience of description, and the identification code does not describe the order of each step, but each step may occur in a different order than the explicitly specified order unless the context clearly indicates a specific order. That is, each step may occur in the same order as specified, may be substantially accommodated at the same time, or may be performed in a reverse order.

All terms used herein have the same meaning as commonly understood by one of ordinary skill in the art to which the present disclosure pertains, unless defined otherwise. Terms defined in general used in the dictionary should be interpreted as being consistent with the meaning in the context of the related technology, and they should not be construed as having an ideal or overly formal meaning unless explicitly defined in the present disclosure.

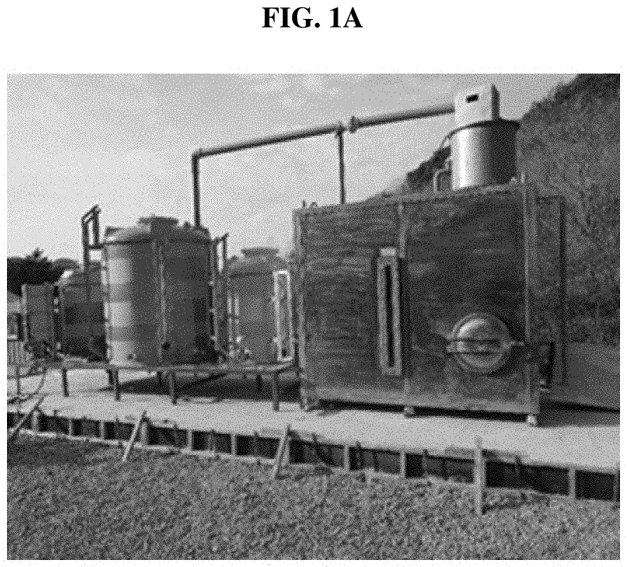

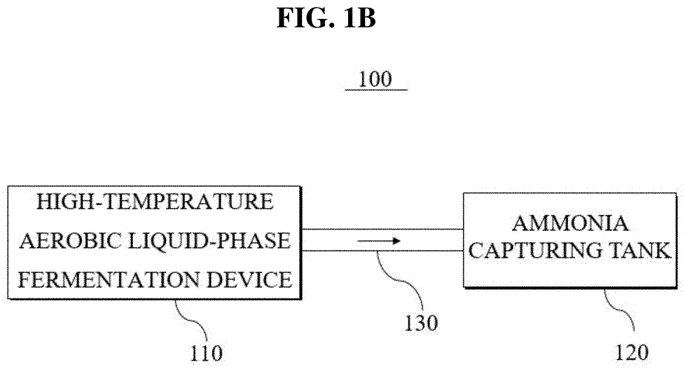

FIGS. 1A and 1B show drawings illustrating a liquid fertilizer production system based on the liquid fertilizer quality certification (LFQC) of livestock manure according to an embodiment of the present disclosure, in which FIG. 1A shows an image illustrating the actual system installed and FIG. 1B shows a schematic block diagram illustrating FIG. 1A.

Referring to FIGS. 1A and 1B, a liquid fertilizer production system 100 includes a high-temperature aerobic liquid-phase fermentation device 110, an ammonia capturing tank 120, and a pipe 130 connecting them. In particular, the liquid fertilizer production system 110, although schematically shown, its specific configuration may be based on a high-temperature aerobic fermentation device disclosed in Korean Patent No. 10-0747682 previously filed by the inventors of the present disclosure and a high-temperature liquid fermentation device disclosed in Korean Patent No. 10-0877588.

The high-temperature aerobic liquid-phase fermentation device 110 is operated such that a liquid fertilizer of livestock manure is accommodated therein, and an appropriate amount of air is supplied to the accommodated liquid fertilizer so as to induce an autothermal reaction to occur by thermophilic microorganisms, and thereby a treatment process of high-temperature aerobic liquid-phase fermentation is performed. In an embodiment, the high-temperature aerobic liquid-phase fermentation device 110 undergoes a process, in which when a liquid fertilizer of livestock manure is injected into a reaction tank made of SUS 304 with a size of 2.5×1.6×2.7 (m) with a treatment capacity of 5 tons, aeration is induced by supplying air to the liquid fertilizer in the reactor. In particular, in the high-temperature aerobic liquid-phase fermentation device 110, the ejectors installed inside the reaction tank are driven by submersible motors of 5 HP output to generate bubbles from the injected air.

In an embodiment, the high-temperature aerobic liquid-phase fermentation device 110 is provided with a defoaming means as a means for removing the bubbles generated due to the injection of air in an upper part of the reaction tank. In particular, the defoaming means includes motors and rotary blades in the form of a propeller, which are connected to each of the motors to rotate. The high-temperature aerobic liquid-phase fermentation device 110 drives these motors of the defoaming means when bubbles are generated inside the reaction tank due to the air injection so as to rotate the rotor blades at 1,780 rpm. In particular, the high-temperature aerobic liquid-phase fermentation device 110 removes bubbles as the blades are rotated by the operation of the motors.

In an embodiment, the high-temperature aerobic liquid-phase fermentation device 110 is connected to an outlet for discharging the ammonia gas generated in the upper part of the defoaming means. The high-temperature aerobic liquid-phase fermentation device 110 discharges the ammonia gas generated through the outlet.

The ammonia gas discharged from the high-temperature aerobic liquid-phase fermentation device 110 is brought into contact with water in the ammonia capturing tank 120 connected to the pipe 130 to absorb and recover ammonia. In particular, phosphoric acid having a purity of 85% is added to the ammonia capturing tank 120 at 1% of the treatment capacity to adjust the pH in the ammonia capturing tank 120 to about pH 3 to about pH 4.

Through a subsequent treatment process based on the liquid treated by the high-temperature aerobic liquid-phase fermentation, a liquid fertilizer suitable for the proposed standards of liquid fertilizer quality certification (LFQC) of livestock manure can be produced. In particular, the proposed standards of liquid fertilizer quality certification (LFQC) of livestock manure may be represented as shown in Table 1 below.

TABLE 1
Standard criteria of official standard of commercial fertilizer and Proposed
LFQC_1 and LFQC_2 check list for inspection of liquid manure fertilizer's quality

| | | | | Official | Premium | | | | | |
|---|---|---|---|---|---|---|---|---|---|---|
| | | | | standard of | liquid | Premium Liquid Fertilizer scoring | | | | |
| | | | | commercial | Fertilizer | system (LFQC_2), Total 25 point | | | | |
| Category | Items | | | fertilizer | (LFQC_1) | 1 point | 2 point | 3 point | 4 point | 5 point |
| Nutrient | 1 | NPK (Total) | % | 0.3% (or | 0.3 (or | <0.35 | 0.35~ | 0.48~ | 0.45~ | 0.50> |
| contents | | | | more) | more) | | 0.40 | 0.45 | 0.50 | |
| | 2 | N | mg/L | — | Components | <500 | 500~ | 1000~ | 1500~ | 2000> |
| | 3 | P | mg/L | — | Components | | 1000 | 1500 | 2000 | |
| | 4 | K | mg/L | — | Components | | | | | |
| Hazardous | 5 | As | mg/kg | 5 | 5 | | | | | |
| contents: | 6 | Cd | mg/kg | 0.5 | 0.5 | | | | | |
| heavy metals | 7 | Hg | mg/kg | 0.2 | 0.2 | | | | | |
| | 8 | Pb | mg/kg | 15 | 15 | | | | | |
| | 9 | Cr | mg/kg | 30 | 30 | | | | | |
| | 10 | Cu | mg/kg | 50 | 50 | | | | | |
| | 11 | Zn | mg/kg | 130 | 130 | | | | | |
| | 12 | Ni | mg/kg | 5 | 5 | | | | | |
| Hazardous | 13 | E. coil O157:H7 | | N/D | N/D | | | | | |
| contents: | 14 | Salmonella | | N/D | N/D | | | | | |
| pathogens | 15 | Staphylococcus Aureus | | — | N/D | | | | | |
| | 16 | Listeria Monocytogenes | | — | N/D | | | | | |
| | 17 | Bacillus Cereus | | — | N/D | | | | | |
| Antibiotics | 18 | Tetracycline | | — | N/D | | | | | |
| | 10 | Beta-Lactam | | — | N/D | | | | | |
| | 20 | Sulfamide | | — | N/D | | | | | |
| | 21 | Macrolide | | — | N/D | | | | | |
| | 22 | Aminoglycoside | | — | N/D | | | | | |
| Maturity & | 23 | Mechanical Stability | | — | Matured | | | | | |
| | | Analysis | | | | | | | | |
| stability | 24 | *LFGI | | — | 70> | <80 | 80~85 | 85~90 | 90~95 | ↑95 |
| Physical | 25 | NaCl | % | ↓0.3% | ↓0.3% | | | | | |
| properties | 28 | Moisture content | % | 95%> | 95%> | | | | | |
| | 27 | Total solids (TS) | % | — | Component | ↑2.0 | 2.0~1.5 | 1.5~1.8 | 1.8~0.5 | <0.5 |
| | 28 | Electrical Conductivity | mS/cm | — | Component | ↑25 | 25~20 | 20~15 | 15~10 | <10 |
| | | (EC) | | | | | | | | |
| | 29 | pH | | — | Component | | | | | |
| | 30 | Odor | | — | Odor | | | | | |
| | | | | | Intensity <1 | | | | | |

↓: below,
↑: over,
<: less than,
>: more than,
∧: above,
∨ :under,
*LFGI = liquid Fertilizer Germination Index

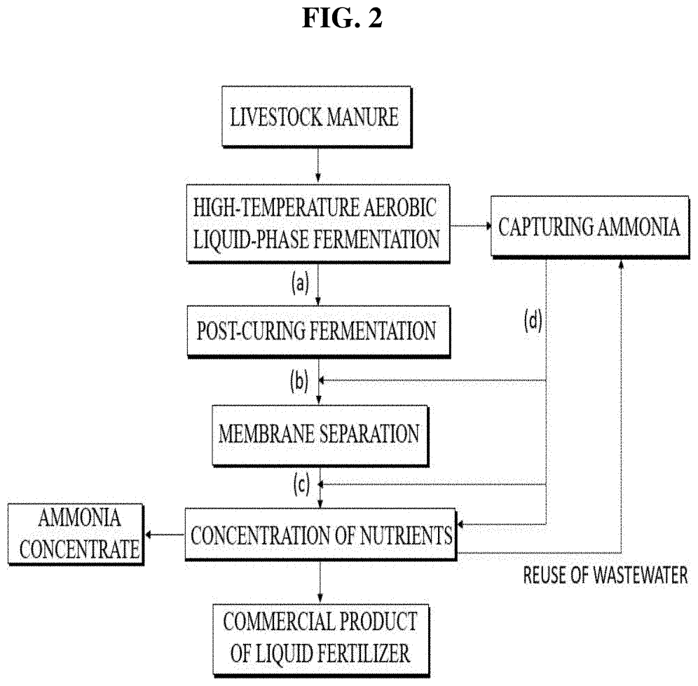

FIG. 2 shows a chart illustrating a process for producing a liquid fertilizer based on the liquid fertilizer quality certification (LFQC) of livestock manure according to an embodiment of the present disclosure.

Referring to FIG. 2, a liquid fertilizer of livestock manure is injected into the reaction tank of the high-temperature aerobic liquid-phase fermentation device 110. In particular, the livestock manure may correspond to a fermentation liquid fertilizer (hereinafter referred to as “released liquid fertilizer”) that is released from a livestock manure joint resource recycling facility.

As a first step to convert livestock manure into a high-quality liquid fertilizer, the high-temperature aerobic liquid-phase fermentation device 110 subjects the liquid fertilizer injected into the reactor to high-temperature aerobic liquid fermentation. In particular, the ammonia gas generated is captured in the ammonia capturing tank 120. The water used to capture the ammonia gas in the ammonia capturing tank 120 may be reused as the waste water used in the downstream process.

As a second step to convert livestock manure into a high-quality liquid fertilizer, the liquid treated by the high-temperature aerobic liquid-phase fermentation is subjected to post-curing fermentation. In particular, the post-curing fermentation is performed such that 5 L/m³·min of air is injected into the liquid (a) treated by the high-temperature aerobic liquid-phase fermentation and aerated for about 14 days. In particular, a fermentation liquid fertilizer that complies with the proposed standards of liquid fertilizer quality certification (LFQC) of livestock manure can be obtained by subjecting the liquid (a) treated by the high-temperature aerobic liquid-phase fermentation to the post-curing fermentation.

As a third step to convert livestock manure into a high-quality liquid fertilizer, the post-cured liquid fertilizer is subjected to the treatment by a separation membrane (UF). In particular, the treatment by a separation membrane may correspond to a physical purification process in a membrane separation device using an ultrafiltration membrane (UF). The specifications of the membrane separation device are shown in Table 2 below.

TABLE 2

| U/F Pump | Material | STS | |
|---|---|---|---|
| | Capacity | 1 TPH × 500M × | |
| | | 0.75 KW | |
| Cartridge | Material | Polypropylene (PP) | |
| Filter | Pore Size | 5 | μm |

| | | |
|---|---|---|
| UF | Material of Module | ABS cap + ABS pipe |
| | Length of Module | φ6″ × 1,120 mm |

| Port Size of Module | 40 | A |
|---|---|---|
| Type of Membrane | Hollow Fiber | |
| | Membrane | |
| Pore Size of Membrane | 0.05 | μm |

| | |
|---|---|
| Net Amount of | 450 to 650 |
| Penetration (LMH) | |
| Maximum Membrane | 3 |
| Potential (kg/cm²) | |
| pH Range | 2 to 13 |

By subjecting the fermentation liquid fertilizer to the separation membrane treatment, the solid particles can be reduced and thereby a high-quality liquid fertilizer with an improved level can be obtained.

Finally, the liquid fertilizer is commercialized by nutrient concentration (R/O) treatment.

### EXAMPLE 1

In the case of the liquid fertilizer released from the common livestock manure resource facility located in area G, it was found as a result of the analysis of physicochemical properties that the liquid fertilizer did not meet the standards of the current fertilizer process specifications of “fermentation liquid of livestock manure”. As can be seen in Table 3 below, it was found that the zinc (Zn) item exceeded the standards.

TABLE 3

| | | | | | | | | Liquid A + | | |
|---|---|---|---|---|---|---|---|---|---|---|
| | | | | | | High-Temperature | | Post-Curing | | Liquid |
| | | | | | | Aerobic Liquid- | | Fermentation | | B + NH₃ |
| | | | | Fermentation | | Phase Fermentation | | for 14 | | Captured |
| | | | | Liquid of | Premium | 0 day | 8 days (= | days (= | Liquid | Solution |
| | | | | Livestock | Liquid | (released | liquid A) | liquid B) | A + UF | (1:1) |
| | | | | Manure | Fertilizer | liquid | (treatment | (treatment | (treatment | (treatment |
| Category | Item | | | (Current) | (LFQC_1) | fertilizer) | group: a) | group: B) | group: c) | group: d) |
| Content | 1 | Total amount | (%) | 0.3% or | 0.3 or | 0.73 | 0.61 | 0.71 | 0.31 | 0.91 |
| according to | | of NPK | | more | greater | | | | | |
| Specification | 2 | N | (mg/L) | — | Component | 3,705 | 2,735 | 2,031 | 1,130 | 4,188 |
| | 3 | P | (mg/L) | | Component | 412 | 271 | 343 | 12 | 2,952 |
| | 4 | K | (mg/L) | — | Component | 3,185 | 3,101 | 4,705 | 2,001 | 943 |
| Maximum | 5 | As | (mg/kg) | 5 | 5 | N/D | N/D | N/D | N/D | N/D |
| Amount of | 6 | Cd | | 0.5 | 0.5 | N/D | N/D | N/D | N/D | N/D |
| Hazardous | 7 | Hg | | 0.2 | 0.2 | N/D | N/D | N/D | N/D | N/D |
| Contents | 8 | Pb | | 15 | 15 | 0.6 | 0.7 | N/D | 0.6 | N/D |
| | 9 | Cr | | 30 | 30 | 2.1 | 1.5 | 1.5 | 1.9 | N/D |
| | 10 | Cu | | 50 | 50 | 36.6 | 23.5 | 30.3 | 9.2 | 9.0 |
| | 11 | Zn | | 130 | 130 | 137.7 | 85.0 | 114.8 | 15.0 | 23.8 |
| | 12 | Ni | | 5 | 5 | 1.1 | 0.9 | N/D | 0.9 | N/D |
| | 13 | E. coli O157: H7 | — | N/D | N/D | — | — | — | — | — |
| | 14 | Salmonella | — | N/D | N/D | — | — | — | — | — |
| | 15 | Staphylococcus | — | — | N/D | — | — | — | — | — |
| | | aureus | | | | | | | | |
| | 16 | Lbaciisteria | — | — | N/D | — | — | — | — | — |
| | | monocytogenes | | | | | | | | |
| | 17 | Bacillus cereus | — | — | N/D | — | — | — | — | — |
| | 18 | Tetracycline | — | — | N/D | — | — | — | — | — |
| | 19 | Beta-Lactam | — | — | N/D | — | — | — | — | — |
| | 20 | Sulfamide | — | — | N/D | — | — | — | — | — |
| | 21 | Microlide | — | — | N/D | — | — | — | — | — |
| | 22 | Aminoglyceride | — | — | N/D | — | — | — | — | — |
| Other | 23 | Maturity | | | | | | | | |
| Specifications | | Measurement | — | — | Post- | Semi- | Semi- | matured | — | — |
| | | Device | | | maturity | matured | matured | | | |
| | 24 | Liquid | — | — | 70% or | 0 | 27 | 80 | 72 | 1 |
| | | Fertilizer | | | more | | | | | |
| | | Germination | | | | | | | | |
| | | Index (LFGI) | | | | | | | | |
| | 25 | NaCl | (%) | 0.3% or | 0.3% or | 0.19 | 0.18 | 0.22 | 0.12 | 0.09 |
| | | | | less | less | | | | | |
| | 26 | Moisture Content | (%) | 95% or | 95% or | 97.4 | 97.6 | 97.7 | 99.1 | 98.2 |
| | | | | more | more | | | | | |
| | 27 | Total Solids (TS) | (%) | — | Component | 2.6 | 2.4 | 2.3 | 0.9 | 1.8 |
| | 28 | Electrical | (mS/cm) | — | Component | 25.6 | 18.3 | 17.3 | 12.6 | 23.0 |
| | | Conductivity (EC) | | | | | | | | |
| | 29 | pH | — | — | Component | 8.7 | 9.3 | 9.3 | 9.4 | 8.8 |
| | 30 | Odor | — | — | Odor | — | — | — | — | — |
| | | | | | intensity: | | | | | |
| | | | | | 1 or smaller | | | | | |

| | | | | | |
|---|---|---|---|---|---|
| Fermentation Liquid of Livestock Manure (Current) | inappropriate | appropriate | appropriate | — | — |
| Premium Liquid Fertilizer (LFQC_1) | inappropriate | inappropriate | appropriate | — | — |
| Premium Liquid Fertilizer Scoring System (LFQC_2) | (13) | (15) | 16 | — | — |

*(N/D: Not Detected)
*In the case of Odor on Item No. 30, the odor intensity in all of the treated groups was assume to be 1 or smaller.

In the case of treated group (a), in which the liquid released from livestock manure was subjected to high-temperature aerobic liquid-phase fermentation, the liquid fertilizer was suitable for the standards of “fermentation liquid of livestock manure” but was not suitable for the standards of liquid fertilizer quality certification (LFQC) of livestock manure.

In the case of treated group (b), in which the liquid treated by the high-temperature aerobic liquid-phase fermentation was subjected to post-curing fermentation, the liquid fertilizer produced was suitable for the standards of liquid fertilizer quality certification (LFQC) of livestock manure.

In the case of treated group (c), in which the liquid treated by the high-temperature aerobic liquid-phase fermentation was subjected to post-curing fermentation and then subjected to a separation membrane treatment, the liquid produced therefrom not only complied with the standards of liquid fertilizer quality certification (LFQC) of livestock manure, but also, due to the reduction of solid particles (e.g., TS, SS, etc.) therein, resulted in production of a high-quality liquid fertilizer which is capable of controlling the phenomenon of blockage of the pipes, etc. at the time of fertigation or nutriculture.

In the case of treated group (d), in which the treatment was made by a 1:1 mixture of the liquid treated by the high-temperature aerobic liquid-phase fermentation and subsequent post-curing fermentation and an ammonia captured solution, the resultant not only complied with the standards of liquid fertilizer quality certification (LFQC) of livestock manure, but also resulted in the production of a high-concentration-nitrogen-enriched liquid fertilizer in which the nitrogen concentration is increased one level higher using the ammonia gas, which is produced from the process of treatment by high-temperature aerobic liquid-phase fermentation, recovered by capturing with phosphoric acid.

While the fermentation liquid fertilizers used in Korea have difficulties in distribution due to their low nitrogen and phosphoric acid concentrations, the product of the present disclosure can increase the distribution efficiency of the liquid fertilizer by significantly increasing the nitrogen and phosphoric acid concentrations by using a nitrogen concentrate (containing phosphoric acid).

Meanwhile, microalgae are algae with a size of about 2 μm to about 50 μm, and about 100,000 species are known thus far, and they are basic producers of photosynthesis. That is, they utilize water, light, carbon dioxide, and nutrients to produce organic matter and oxygen.

Industrially, microalgae are used in the production of biodiesel, food and food additives, feed and fertilizer, pharmaceuticals, etc. For the industrial utilization of microalgae including agriculture, cultivation using the growth characteristics of microalgae is essential, and the cultivation of microalgae includes a closed-type culture and an open-type culture according to the culture type, and a fed-batch culture and a continuous culture according to the culture method.

Carbon and inorganic salts are required for high-density culture, and the amount of supply and the ratio of nitrogen and phosphorus contained therein are important factors for the growth of microalgae. Additionally, as in terrestrial plants that perform photosynthesis, the growth of microalgae is greatly affected by temperature, amount of light, and quality of light.

Agricultural utilization of microalgae includes its use as a by-product for livestock feed, aquaculture feed, fertilizers, and soil improvement, but is not limited thereto, and it can also be used for complex farming in which aquatic production and crop production are combined. Among microalgae, those of the genus Chlorella have a high industrial use value such as food, and many studies have been conducted on their biological properties. Recently, as microalgae of the genus Chlorella are known to have a positive effect on the production of agricultural crops, there is a growing demand for the development of methods of utilizing the same.

Among microalgae, Chlorella is a kind of freshwater algae. Since they contain proteins, chlorophylls, vitamins, minerals, nucleic acids, and unsaturated fatty acids in cells, they have been reported to be only nutritionally excellent, and can also improve various physiological activities (e.g., improvements of immune functions, antioxidative functions, liver functions, etc.).

The main components of Chlorella include 50-60% of crude protein, 15-20% of carbohydrate, and 12-18% of crude lipid, in particular, in which about 30% of the lipid is linolein, about 15% is palmitic acid, and the carbohydrate contains a large amount of hemicellulose, thus making Chlorella suitable as a functional food material and is being used as aquaculture feed for juvenile fish in the aquaculture industry.

Chlorella is also called a future food because it contains essential nutrients for the human body in a balanced way and has been recognized as a new biological resource (biomass) due to its ability to store proteins or lipids (bio-diesel). Chlorella has been reported that Chlorella suppresses weed germination and promotes rice growth when treated during cultivation of rice.

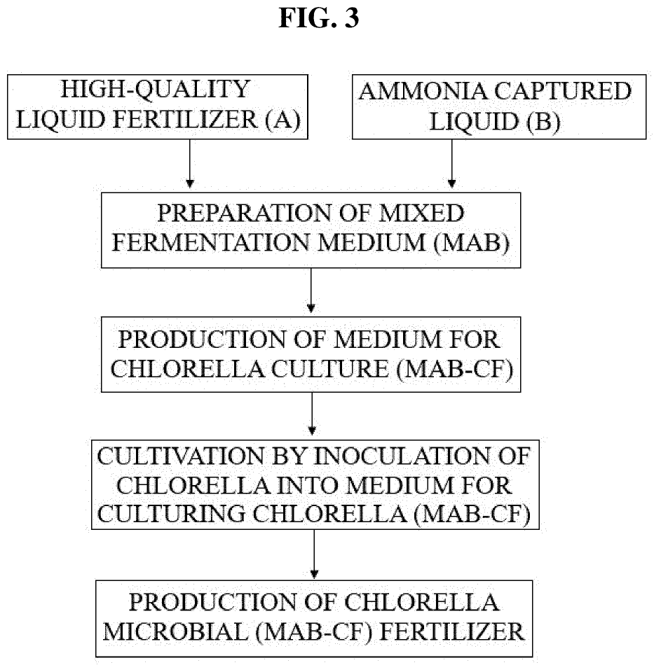

FIG. 3 shows a chart illustrating the process of preparing a Chlorella microbial fertilizer using the high-quality liquid fertilizer produced in FIG. 2.

Referring to FIG. 3, a mixed fermentation medium (MAB) is prepared by appropriately mixing a high-quality liquid fertilizer with an improvement of one level in quality (hereinafter, “liquid A”), which complies with the standards of liquid fertilizer quality certification (LFQC) of livestock manure through a process of high-temperature aerobic liquid-phase fermentation, a process of post-curing fermentation, and a process of a separation membrane treatment while reducing solid particles among the processes for producing a liquid fertilizer shown in FIG. 2, and an ammonia captured solution (hereinafter, “solution B”), in which ammonia generated during the process of high-temperature aerobic liquid-phase fermentation is captured, are appropriately mixed.

In an embodiment, the ammonia captured solution (solution B) is captured such that the ammonia generated during the process of high-temperature aerobic liquid-phase fermentation is captured with the phosphoric acid in the ammonia capturing tank 120.

The prepared mixed fermentation medium (MAB) is diluted to produce a Chlorella culture medium (MAB-CF) suitable for Chlorella culture. In particular, the dilution factor may be based on the dilution factor calculation method suitable for a liquid fertilizer disclosed in Korean Patent Registration No. 10-1859167 previously filed by the inventors of the present disclosure. CHLORELLA culture medium (MAB-CF) refers to a titrated a mixed fermentation medium for culturing Chlorella.

A Chlorella-containing microbial fertilizer (MAB-CF-16) can be prepared by inoculating and culturing Chlorella using the organic and inorganic nutrient sources of the produced Chlorella culture medium (MAB-CF).

In an embodiment, the mixed fermentation medium, which is prepared by mixing the high-quality liquid fertilizer (solution A) and the ammonia captured solution (solution B), can provide the effect of reducing the production cost of crops by replacing the high-cost Chlorella producing chemical medium. In particular, the physicochemical properties of each of the high-quality liquid fertilizer (solution A) and the ammonia captured solution (solution B) used are shown in Table 4 below.

TABLE 4
Properties of high-quality liquid fertilizer (A) and ammonia captured solution (B)

| | N | NH₄—N | NO₃—N | P | K | E. coli |
|---|---|---|---|---|---|---|
| Item | (mg/kg) | (mg/kg) | (mg/kg) | (mg/kg) | (mg/kg) | (CFU/mL) |
| High-Quality Liquid | 1,200 ± 30 | 818 ± 50 | 143 ± 25 | 14 ± 1 | 3421 ± 15 | Not detected |
| Fertilizer (A) | | | | | | |
| Ammonia Captured | 4,280 ± 3 | 4,200 ± 10 | 43 ± 25 | 14 ± 1 | 3421 ± 15 | Not detected |
| Solution (B) | | | | | | |

Meanwhile, the Chlorella culture medium (MAB-CF) produced in an embodiment is a mixed fermentation medium, which is prepared by appropriately mixing the high-quality liquid fertilizer (solution A) and the ammonia captured solution (solution B), was prepared at a T-N concentration similar to that of the expensive BG11 medium widely used as a Chlorella culture medium, and the comparison of the properties is shown in Table 5 below.

TABLE 5
Properties of Mixed medium (MAB-CF) and BG11 medium

| | N | NH₄—N | NO₃—N | P | K | E. coli |
|---|---|---|---|---|---|---|
| Item | (mg/kg) | (mg/kg) | (mg/kg) | (mg/kg) | (mg/kg) | (CFU/mL) |
| BG11 (Cont.) | 247 | Not detected | 247 | 4.68 | 13.7 | Not detected |
| Mixed Medium (MAB-CF) | 270 ± 5 | 250 ± 1 | 9 ± 1 | 1,010 ± 1 | 174 ± 1 | Not detected |

An outdoor photo-culture reactor for culturing Chlorella was prepared based on a 12 mm thick circle shape made of a transparent acrylic material to improve light transmittance. The total culture size was 200 L and the reactor was installed and operated on the immediately right side of a container device for the fermentation liquid fertilizer process.

In an embodiment, a Chlorella microbial fertilizer (MAB-CF-16) can be produced by inoculating a prepared mixed fermentation medium and Chlorella.

1) Experiment 1: Effect of Chlorella production using mixed fermentation medium for Chlorella culture (MAB-CF)

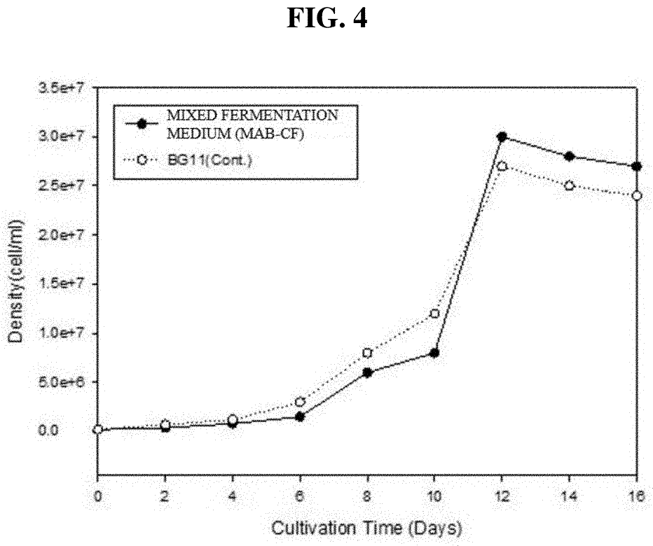

In the present disclosure, the initial concentration of Chlorella inoculation was 1×10⁴ cell/mL, and Chlorella was inoculated into BG11 medium and a mixed fermentation medium at the same concentration. On the 16th day of culture, as shown in FIG. 4, the concentration was shown to be 2.7×10⁷ cells/mL in the mixed fermentation medium, and 2.4×10⁷ cells/mL in BG11 medium, which is an expensive chemical medium.

As a result, a rather higher density of Chlorella bacteria cell number (nutrient cells) was shown in the mixed fermentation medium (MAB-CF), and thus it can sufficiently replace the expensive chemical medium.

2) Experiment 2: Results of outdoor culture and seed germination experiment in mixed fermentation medium for Chlorella culture (MAB-CF)

The growth potential of Chlorella was examined by installing a photo-culture reactor for Chlorella outdoors and a seed germination experiment was performed to examine the stability of Chlorella when used as a liquid fertilizer.

### Review of Stability through Seedless Germination Experiments

A seed germination experiment was performed using a Chlorella containing microbial fertilizer (MAB-CF-16) cultured in a mixed fermentation medium (MAB-CF) for 16 days and a Chlorella BG11-16 cultured in a chemical medium BG11-16 for 16 days were subjected to a seed germination experiment using radish seeds. The results are shown in Table 6 below.

TABLE 6
Seedless germination experiments of chlorella microbial
fertilizer (MAB-CF-16) and BG11-16 (n = 3)

| | | | Chlorella |
|---|---|---|---|
| | No | | Microbial |
| | treatment | | Fertilizer |
| Item | (Cont.) | BG11-16 | (MAB-CF-16) |
| Germination rate (%) | 90 | 98 | 98 |
| Root Length (average) | 6 | 6.82 | 7.99 |
| Germination rate (%) | 100 | 111 | 112 |
| Root Length | 100 | 120 | 140 |
| Germination Index (GI) | 100 | 133 | 154 |

This experiment showed that the Chlorella microbial fertilizer (MAB-CF-16) had a higher germination index than that of a chemical medium BG11-16 thus being stable.

Since the Chlorella microbial fertilizer (MAB-CF-16: a Chlorella microbial liquid fertilizer) has various functions (e.g., improving antioxidant activity, detoxification of pesticides and heavy metals, etc.), it has a very high potential for use in environment-friendly agriculture. In addition, Chlorella has been reported to have the effects of promoting crop growth, improving a storage property, and improving a sugar content, it is expected to contribute to quality improvement such as promoting crop growth when Chlorella is used in agricultural fields by developing a practical technology using the biological characteristics of Chlorella.

### Review of Stability Microbial Fertilizer through Immersion Test of Pepper Seed

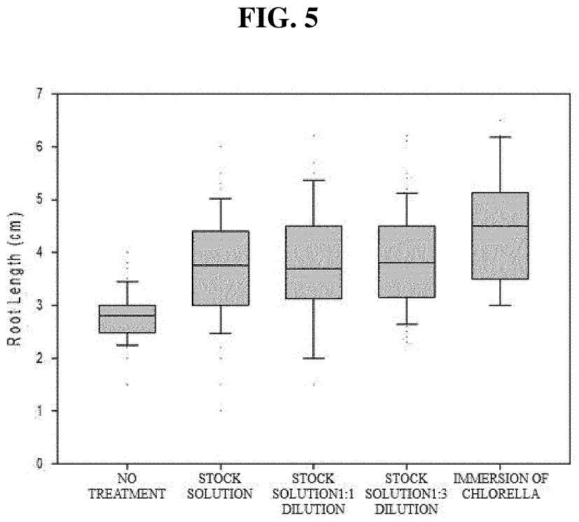

An experiment was conducted to examine the effect of a Chlorella microbial fertilizer on the germination of pepper seeds. The root length was measured for a case of no treatment group where a pepper seed was allowed to germinate in general water, a case where a pepper seed was allowed to germinate by diluting a chlorella microbial fertilizer with water, and a case where a pepper seed was allowed to germinate after immersion of the pepper seed before its germination, and the results are as shown in FIG. 5. Referring to FIG. 5, in relation to the germination of pepper seeds, the most dominant root length was shown in the case where a pepper seed was allowed to germinate after its immersion with a Chlorella microbial fertilizer. Therefore, when a pepper seed germinates after immersion with a Chlorella microbial fertilizer before germination, it can be more developed to have more developed roots.

### Effects of Chlorella Microbial Fertilizer on Sugar Content of Strawberries and Chlorella of Leaves

A cultured Chlorella microbial fertilizer was diluted with groundwater in 5 experimental groups among the 90 m lines in a farmhouse growing organic strawberries (variety: Sulhyang), and after the application of the fertilizer on foliar surfaces once a week for a total of 4 weeks, chlorophyll of strawberry leaves and sugar content of strawberries were measured.

The sugar content was measured 3 times repeatedly using a portable saccharimeter (Palm Abbe 203, MISCO, USA), 15 per treatment group, and the measurement results are shown in Table 7 below.

TABLE 7
Measured values of sugar content (brix) of strawberry “Sulhyang” according
to groups treated with chlorella microbial fertilizer (MAB-CF-16)

| | Control | Control | MAB-CF- | MAB-CF- | MAB-CF- | MAB-CF- | MAB-CF- | MAB-CF- |
|---|---|---|---|---|---|---|---|---|
| Treatment | Group 1 | Group 2 | 16 × 25 | 16 × 50 | 16 × 100 | 16 × 250 | 16 × 500 | 16 × 1,000 |
| Repeat 1 | 10.0 | 9.0 | 9.0 | 11.0 | 12.0 | 9.0 | 10.0 | 9.0 |
| Repeat 2 | 9.5 | 10.0 | 9.0 | 8.0 | 8.5 | 11.5 | 10.0 | 9.0 |
| Repeat 3 | 9.5 | 8.0 | 12.0 | 11.0 | 9.0 | 9.0 | 10.0 | 9.0 |
| Average | 9.67 | 9.0 | 10.3 | 10.5 | 9.83 | 9.7 | 10.0 | 9.0 |

MAB-CF-16 = chlorella microbial fertilizer produced for 16 days

As shown in Table 7 above, when the group, where the folial surfaces were treated with Chlorella, and no treatment group were compared, it was confirmed that the sugar content of the strawberry variety “Sulhyang” was improved by 12.1% in the group treated with a 25-fold dilution of Chlorella microbial fertilizer (MAB-CF-16), and showed a sugar content (brix) of 9.0, which was the lowest sugar content, in the group treated with a 1,000-fold dilution of Chlorella microbial fertilizer (MAB -CF-16).

Therefore, it was speculated that the foliar application of Chlorella microbial fertilizer (MAB-CF-16) could increase the sugar content of strawberries “Seolhyang”, and that 25- to 100-fold dilution would be the optimum concentration to be applicable.

The chlorophyll (SPAD) measurement was averaged after performing three repeated measurements of five random leaves for each experimental group. As a result of the chlorophyll (SPAD) measurement, the Chlorella microbial fertilizer (MAB-CF-16) showed the highest value in the group treated with a 250-fold dilution.

TABLE 8
Measured values of chlorophyll on foliar surfaces of strawberry “Sulhyang”
according to treatment groups with chlorella microbial fertilizer (MAB-CF-16)

| | Control | MAB-CF- | MAB-CF- | MAB-CF- | MAB-CF- | MAB-CF- | MAB-CF- |
|---|---|---|---|---|---|---|---|
| Treatment | Group | 16 × 25 | 16 × 50 | 16 × 100 | 16 × 250 | 16 × 500 | 16 × 1,000 |
| Repeat 1 | 46.1 | 37.7 | 41.6 | 43.7 | 47.2 | 41.2 | 42.8 |
| Repeat 2 | 42.4 | 35.9 | 42.4 | 41.8 | 46.4 | 44.8 | 43.4 |
| Repeat 3 | 43.2 | 32.3 | 37.4 | 42.8 | 43.2 | 44.2 | 35.0 |
| Average | 43.9 | 35.3 | 40.47 | 42.77 | 45.6 | 43.4 | 40.4 |

*MAB-CF-16 = chlorella microbial fertilizer produced for 16 days

No strawberry damage was found in the strawberry application test of the Chlorella microbial fertilizer (MAB-CF-16), and when compared with the control (tolerance), it was found that the fertilization at a 50-fold dilution concentration was preferable in order to achieve the effects of increasing root length, sugar content, and chlorophyll.

Although the present disclosure has been described above with reference to preferred embodiments, those skilled in the art will understand that various modifications and changes can be made to the present disclosure without departing from the spirit and scope of the present disclosure described in the claims below.

As described above, the method for producing a liquid fertilizer based on liquid fertilizer quality certification (LFQC) of livestock manure, a high-quality liquid fertilizer produced through the method, and the method for preparing a Chlorella microbial fertilizer can be utilized in industrial fields related to livestock manure recycling.

1. A method for producing a liquid fertilizer based on liquid fertilizer quality certification (LFQC) of livestock manure, comprising:
subjecting a liquid released from livestock manure to high-temperature aerobic liquid-phase fermentation;
subjecting the liquid resulting from the high-temperature aerobic liquid-phase fermentation to post-curing fermentation to produce a fermentation liquid fertilizer that complies with the standards of liquid fertilizer quality certification (LFQC) of livestock manure; and
subjecting the produced fermentation liquid fertilizer to a separation membrane to produce a high-quality liquid fertilizer with reduced solid particles.

2. The method of claim 1, further comprising a step of bringing the ammonia gas, which is produced in the step of the high-temperature aerobic liquid-phase fermentation, into contact with water in an ammonia capturing tank to thereby capture ammonia.

3. The method of claim 2, wherein the step of capturing ammonia gas is characterized in that phosphoric acid with a purity of 85% is injected into the ammonia capturing tank at 1% of a treatment capacity, and thereby the ammonia gas is recovered by capturing and concentrating with a phosphoric acid solution.

4. The method of claim 3, further comprising a step of mixing the fermentation liquid fertilizer and the ammonia captured solution at a 1:1 ratio to thereby produce a liquid fertilizer in which high-concentration nitrogen is enriched.

5. A high-quality liquid fertilizer produced by the method for producing a liquid fertilizer according to claim 1.

6. A method for preparing a Chlorella microbial fertilizer, comprising:
subjecting a liquid released from livestock manure to high-temperature aerobic liquid-phase fermentation, and capturing the ammonia gas produced in the step of the high-temperature aerobic liquid-phase fermentation;
subjecting the liquid resulting from the high-temperature aerobic liquid-phase fermentation to post-curing fermentation, and subjecting the produced fermentation liquid fertilizer to a separation membrane to produce a high-quality liquid fertilizer that complies with the standards of liquid fertilizer quality certification (LFQC) of livestock manure;
mixing the high-quality liquid fertilizer produced and the ammonia captured solution to prepare a mixed fermentation medium; and
culturing Chlorella in the mixed fermentation medium to prepare a Chlorella microbial fertilizer.

7. The method of claim 6, wherein the step of preparing the mixed fermentation medium is characterized in that the prepared mixed fermentation medium is diluted and a medium for culturing Chlorella to a total-nitrogen (T-N) concentration corresponding to BG11 chemical medium is prepared.
